(19) United States
(12) Patent Application Publication
(10) Pub. No.: US 2008/0015546 A1
(43) Pub. Date: Jan. 17, 2008

(54) METHODS AND FORMULATIONS FOR OPTIMAL LOCAL DELIVERY OF CELL THERAPY VIA MINIMALLY INVASIVE PROCEDURES

(76) Inventors: Jesus W. Casas, Brooklyn Park, MN (US); Adam A. Blakstvedt, Big Lake, MN (US); Molly B. Schiltgen, Lake Elmo, MN (US); Kent E. Wika, New Brighton, MN (US); Gyongike M. Molnar, Natick, MA (US)

Correspondence Address: FOX ROTHSCHILD, LLP, 997 LENOX DRIVE, LAWRENCEVILLE, NJ, 08648, US

(21) Appl. No.: 11/776,149

(22) Filed: Jul. 11, 2007

Related U.S. Application Data
(60) Provisional application No. 60/830,455, filed on Jul. 13, 2006.

(51) Int. Cl. A61M 31/00

(52) U.S. Cl. 604/522; 604/93.01

(57) ABSTRACT

The present invention relates to methods, kits, and compositions for safe and efficient delivery of a bioagent to a targeted area of an organ. The method comprises preparing a suspension comprising the bioagent, a contrast agent, and a vehicle, wherein said suspension has an osmolarity from about 270 mOsm to about 440 mOsm; and dispensing at least a portion of said suspension into the targeted area. The invention further provides a kit for delivering a bioagent into a targeted area of an organ comprising: a delivery device; a contrast agent; and a vehicle.

### CROSS-REFERENCED APPLICATION

This application claims priority from U.S. Provisional Application Ser. No. 60/830,455 filed on Jul. 13, 2006. The entirety of that provisional application is incorporated herein by reference.

### FIELD OF THE INVENTION

The present invention relates to formulations, kits and methods for optimal delivery of therapy into organs using minimally invasive means such as catheters.

### BACKGROUND

Myocardial infarction and other pathologic conditions of the heart result in loss of cardiomyocytes, scar formation, ventricular remodeling, and eventually heart failure. Since pharmacologic and interventional strategies fail to regenerate dead myocardium, heart failure continues to be a major health problem worldwide. Dawn B. et al. (2005) Minerva Cardioangiol. 53:549-64. For example, myocardial infarction accounts for approximately 20% of all deaths. It is a major cause of sudden death in adults. U.S. Pat. No. 20040208845. In the U.S., 900,000 people annually suffer from acute myocardial infarction. U.S. Pat. No. 20040253209.

Cell therapy for cardiac repair has emerged as one of the most exciting and promising developments in cardiovascular medicine. Evidence from experimental and clinical studies is increasing that this innovative treatment will influence clinical practice in the future.

Cardiac cell therapy involves transplanting cells into the damaged or diseased myocardium with the goal of repopulating the infarcted areas and restoring the lost contractile function. Research in this field is reviewed in Cellular Cardiomyoplasty: Myocardial Repair with Cell Implantation, ed. Kao and Chiu, Landes Bioscience (1997), particularly Chapters 5 and 8. While the mode of delivery most commonly used in this emerging field is direct myocardial injection, this is needle-based injection into the myocardium during an open chest surgery or direct visualization of the target site. In order for this therapeutic modality to be broadly applied requires a minimally invasive approach for therapy delivery, which is safe, accurate and efficient. Several factors including volumes for delivery, formulations, procedures and ability to monitor ongoing delivery procedures are critical. This invention addresses and discloses these and other improvements.

Ongoing imaging is important for cardiac cell therapy is important to track and verify the placement of the injecting device into the targeted area of the heart and to avoid untoward events such as, for example, damage to vital structures (arteries), or to avoid false delivery into non-targeted sites such as pericardial sac and/or ventricular chamber. Formulations and procedures for incorporating and using contrast agents within bioactive suspensions for therapy of cardiac and other organ disease are described. Likewise, for acute and feasibility research and development, several non-invasive imaging approaches which aim at tracking of transplanted cells in the heart have been used to generate data supporting this invention. Among these are direct labeling of cells with radionuclides or paramagnetic agents, and the use of reporter genes for imaging of cell transplantation and differentiation. Initial studies have suggested that these molecular imaging techniques have great potential. Bengel F M et al., (2005) Eur T Nucl Med Mol Imaging 32, Suppl 2:S404-16.

Studies by others, using either direct or catheter-delivery, indicate low rates of cell retention in target tissues (delivery efficiency). For example, Aicher et al. reports that an injection of endothelial progenitor cells into Left Ventricles of athymic nude rats with myocardial infarction resulted in only 4.7% of the injected cells retained in the heart. Circulation 107: 2134-2139 (2003).

Accordingly, despite the advances recently made in the art, new formulations and methods of targeted cell delivery into the organs in need thereof are needed to better utilize the advantages of cell therapy.

### SUMMARY OF THE INVENTION

One aspect of the present invention provides a method of delivering a bioagent to a targeted area of an organ comprising preparing a suspension comprising the bioagent, a contrast agent, and a vehicle, wherein said suspension has an osmolarity from about 270 mOsm to about 440 mOsm; providing the operator with intra-operative feedback and dispensing at least a portion of said suspension into the targeted area.

In different embodiments of the invention, the bioagent is selected from the group consisting of cells, proteins, drugs, nucleic acids, or a combination thereof.

The cells may be selected from the group consisting of mature myogenic cells (e.g., skeletal myocytes, cardiomyocytes, purkinje cells, fibroblasts), progenitor myogenic cells (such as myoblasts), mature non-myogenic cells (such as endothelial and epithelial cells), hematopoietic cells (monocytes, macrophages, fibroblasts, alpha islet cells, beta islet cells, cord blood cells, erythrocytes, platelets, etc.) or stem cells (pluripotent stem cells, mesenchymal stem cells, endodermal stem cells, ectodermal stem cells, whether adult or embryonic, or whether autologous, allogenic, or xenogenic). More particularly, cells which may be delivered according to this invention further include islet cells, hepatocytes, chondrocytes, osteoblasts, neuronal cells, glial cells, smooth muscle cells, endothelial cells, skeletal myoblasts, nucleus pulposus cells, and epithelial cells.

Another aspect of the present invention provides the method for determining the volume for individual injections, based on anticipated or diagnosed characteristics of the target tissue, such as morphology, structure, and other qualities. The intercellular space can only accommodate a finite amount of fluid or volume/mass, which this is directly dependent on the thickness and inversely dependent on the collagen content (i.e., fibrosis). In different embodiments of the invention, the volume of one injection is between about 10 μl and about 200 μl, with the larger volume preferentially for tissues with high local compliance such as acute infarcts, or inflammatory conditions of organs such as liver, lungs and others. Preferably, the volume of one injection is between about 10 μl and about 160 μl, more preferably about 10 μl and about 80 μl.

In another aspect, the invention provides a method of delivering the bioagent to the targeted area of the organ, wherein a total volume injected into the targeted area is approximately equal to a product of the interstitial capacity and the volume (mass) of the targeted area.

In yet another aspect, the invention provides a kit for delivering a bioagent into a targeted area of an organ comprising a delivery device, a contrast agent, and a vehicle. In one embodiment, a kit is provided, where the bioagent comprises cells. The kit also comprises a freshly prepared contrast agent formulation that contains Isovue and water at predetermined ratios. This formulation is used to resuspend the bioagent to a desired concentration. The resultant therapeutic formulation has 250 to 440 mOsm and optimal imaging properties. In another embodiment, the bioagent comprises a cells suspension at high density (high cell number/mL), at numbers that are specific to cell size thus to cell types. This cell suspension is combined with unmodified and commercially available Isovue or Visipaque at predetermined ratio, such that, the resultant formulation is within 250 and 440 mOsm, good cell compatibility, and with optimal imaging (fluoroscopic) capabilities. In this embodiment, the preparation of the formulation for injection can be done intraoperative without the need for additional equipment, such as centrifuge. In yet another embodiment, a set of instructions is provided with the kit. The instructions contain information necessary or desirable to practice the invention safely and efficiently.

### BRIEF DESCRIPTION OF THE DRAWINGS

FIG. 1 is a graph of top and bottom cell count ratios.

FIG. 2 is a graph of a ratio of top and bottom total cell counts.

FIG. 3 is a graph of myoblast cell settling.

FIG. 4 is a table of catheter values.

FIG. 5 consists of images taken during cell settling.

FIG. 6 is a table of delivery dynamics for cell delivery through catheters.

FIGS. 7A-H are SEM photographs of the inner lumens of catheters.

### DETAILED DESCRIPTION

The current invention fulfills this and other foregoing needs by providing methods, components, and kits for delivery of cell therapy into a targeted area of an organ.

### DEFINITIONS

To aid in the understanding of the invention, the following non-limiting definitions are provided:

The term “allograft” refers to a graft of tissue obtained from a donor of the same species as, but with a different genetic make-up from, the recipient, as a tissue transplant between two humans.

The term “autologous” refers to being derived or transferred from the same individual's body, such as for example an autologous bone marrow transplant.

The term “bioagent” refers to any additive delivered to the targeted area of the organ including but not limited to cells including embryonic and adult stem cells, proteins, drugs, nucleic acids, or a combination thereof. In some embodiments of the invention the bioagent is an adult stem cell derived from brain, bone marrow, peripheral blood, cord blood, blood vessels, skeletal muscle, skin and liver, heart.

The term “contrast agent” refers to a substance that facilitates the X-ray imaging of anatomical structures or compartments that otherwise would be invisible to discriminate.

The term “extraneous genetic material” shall mean any DNA sequence and any RNA sequence which is not originally present within the nuclear genome of a cell.

The term “patient” includes a living or cultured system upon which the methods and/or kits of the current invention is used. The term includes, without limitation, humans.

The term “phenotype” shall mean all properties of an organism, including, without limitation, a cell, except for the genome. As a non-limiting example, expression or quantity of RNA and protein expression, or changes in protein function due to, for example, a mutation, are included within the meaning of the term “phenotype.” Accordingly, changes in expression or quantity of any RNA or protein or changes in activity of any protein in the cell are considered alterations in phenotype of that cell.

The term “practitioner” means a person who is using the methods and/or kits of the current disclosure on the patient. This term includes, without limitation, doctors, other medical personnel, veterinarians, and scientists.

A person skilled in the art will undoubtedly appreciate that at least some part of the prepared suspension will be lost. Accordingly, the term “total volume” refers to the total volume injected to a patient, not a volume of the suspension prepared.

The term “treating” or “treatment” of a disease refers to executing a protocol, which may include administering one or more bioagents to a patient (human or otherwise), in an effort to alleviate signs or symptoms of the disease. Alleviation can occur prior to signs or symptoms of the disease appearing, as well as after their appearance. Thus, “treating” or “treatment” includes “preventing” or “prevention” of disease. In addition, “treating” or “treatment” does not require complete alleviation of signs or symptoms, does not require a cure, and specifically includes protocols which have only a marginal effect on the patient.

The term “xenograft” refers to tissue or organs from an individual of one species transplanted into or grafted onto an organism of another species, genus, or family.

In one embodiment, the organ is an organ or tissue having minimally invasive access, such as, for example, transvascular or endoscopic access. In accordance with this embodiment, non-limiting examples of suitable organs or tissues are a heart, a liver, a kidney, a respiratory tract, a digestive tract, a urinary tract, and in women, a reproductive tract, including a vagina, a uterus, fallopian tubes, and ovaries.

Generally, a targeted area of the organ is an area which is in need of the treatment provided by the bioagent. For example, using cells as the bioagent is appropriate for the targeted areas in need of cellular repopulation. Such need may arise because of multiple reasons, such as, for example, infarction or wounding. In different embodiments of the invention, when the targeted organ is the heart, the targeted area is preferably a myocardial region having a vascular access from which the lesion can be reached for treatment. Regions susceptible for catheter therapy include, without limitations, intraventricular septum, apex, left ventricle free wall, LV lateral, and posterior wall, or any combination thereof.

In one embodiment of the invention, the suspension to be delivered to the targeted area of the organ comprises the bioagent, a contrast agent, and a vehicle.

In one embodiment, the bioagent comprises cells. Preferably, suitable cells are cells which possess the functions of the native cells or cells which can differentiate into suitable cell types. Such cells may include mature myogenic cells (e.g., skeletal myocytes, cardiomyocytes, purkinje cells, fibroblasts), progenitor myogenic cells (such as myoblasts), mature non-myogenic cells (such as endothelial and epithelial cells), hematopoietic cells (monocytes, macrophages, fibroblasts, alpha islet cells, beta islet cells, cord blood cells, erythrocytes, platelets, etc.) or stem cells (pluripotent stem cells, mesenchymal stem cells, endodermal stem cells, ectodermal stem cells, whether adult or embryonic, or whether autologous, allogenic, or xenogenic). More particularly, cells which may be delivered according to this invention further include islet cells, hepatocytes, chondrocytes, osteoblasts, neuronal cells, glial cells, smooth muscle cells, endothelial cells, skeletal myoblasts, nucleus pulposus cells, and epithelial cells. In one embodiment, the cells are at a concentration from about 10×10⁶ per ml to about 300×10⁶ per ml, preferably of up to about 170×10⁶ per ml.

The members of the plurality of cells may be obtained from an autologous source, such as, for example, bone marrow of the patient, from an autograft source, such as, for example, relatives of the patient, or from a xenographic source, preferably, from a member of a close species (for example, if the patient is human, the donor may be a primate, such as, for example, gorilla or chimpanzee). In a preferred embodiment, both the donor and the patient are humans.

A person skilled in the art will undoubtedly appreciate that at least a portion of the cells can be modified, for example, by introducing an extraneous genetic material which, preferably, alters a phenotype of members of at least the portion of the cells. In one embodiment, such extraneous genetic material includes, for example, siRNAs or coding sequences of genes of interest under direction of promoters, which induce expression of the coding sequences. In another embodiment, the extraneous genetic material includes sequences capable of recombination with genomic sequences thus removing selected sequences from cellular genome, which leads to decrease of expression of these removed selected sequences.

The methods of introducing the extraneous genetic material to cells are known to a person of ordinary skill in the art and are reviewed in, for example, Sambrook and Russel, Molecular Cloning: A Laboratory Manual (3^{rd} Edition), Cold Spring Harbor Press, NY, 2000, incorporated herein by reference. These methods include, without limitation, physical transfer techniques, such as, for example, microinjection or electroporation; transfections, such as, for example, calcium phosphate transfections; membrane fusion transfer, using, for example, liposomes; and viral transfer, such as, for example, the transfer using DNA or retroviral vectors.

Another advantage of the current invention is that the invention provides for a real-time intraoperative feedback allowing a practitioner to monitor and optimize the delivery/placement of the suspension into the targeted area/tissue while avoiding rupturing of blood vessels, thus increasing the efficiency and minimizing trauma on the patient and the unnecessary systemic biodistribution of the injectate.

In the generation of pre-clinical data to support and substantiate the invention on optimal methods and procedures for delivery of therapy using minimally invasive catheters, cells were labeled with a marker. Cell markers used include, without limitation, Feridex™ from Berlex Laboratories (Montville, N.J.), europium nanoparticles, available from Biopal (Worcester, Mass.).

The method further provides a contrast agent, capable of providing imaging feedback during ongoing use of the method of the current invention. The imaging feedback may be obtained by such techniques as, for example, MRI and fluoroscopy. The suitable contrast agents include iodine-based contrast agents, such as, for example, iopamidol, commercially available as Isovue™ (Bracco Diagnostics Inc., Princeton, N.J.) or iodixanol, commercially available as Visipaque™ (Nyocomed, Inc., Princeton, N.J.), and gandolinium-based contrast agents, such as, for example, gadodiaminde, commercially available as Omniscan (available from GE Healthcare, Princeton, N.J.). In different embodiments, the contrast agent comprises iopamidol at a concentration of about 25% to about 35%, such as, for example, about 27.6% or about 134 mg/ml. In another embodiment, the contrast agent is iodixanol at a concentration of at least about 145 mg/ml.

It is within the expertise of a person of ordinary skill in the art to interpret the data obtained from the use of the contrast agent. Below are a few non-limiting examples of such interpretation.

For example, if the organ is the heart, the presence of contrast-positive imaging during an injection indicates an optimal intramural myocardial injection. On the other hand, the absence of contrast positive imaging during the injection suggests a false injection, for example, into a ventricular chamber or into a pericardial sac. The absence of contrast positive imaging furthers suggests stopping the ongoing injection and the repositioning of the injecting equipment, such as, for example, a catheter, for a new injection attempt.

The presence of a positive contrast imaging showing a diffuse pattern of local distribution during a given injection indicates a delivery into a tissue site with softer characteristics (e.g., normal tissue, marginal tissue, acute and sub-acute infarcts, non-fibrotic tissue, non-calcified tissue). On the other hand, the presence of contrast positive imaging showing a localized, more defined distribution during a given injection indicates delivery into a tissue site with harder characteristics (e.g., chronic infarct, fibrotic tissue, calcified tissue).

In one suitable embodiment, the suspension comprises about 25% to about 35% v/v of the contrast agent. The suspension further comprises the cells resuspended at about 50% v/v of the composition and has an osmolarity of between about 250 mOsm and about 440 mOsm, preferably between 280 mOsm and 300 mOsm, more preferably from 285 mOsm to 295 mOsm.

Another type of cell delivery medium is that shown in U.S. Pat. No. 5,543,316, which describes an injectable composition comprising myoblasts and an injectable grade medium having certain components designed for maintaining viability of the myoblasts for extended periods of time. The osmolality of the medium is preferably from about 250 mOsm/kg to about 550 mOsm/kg (e.g., more preferably selected from the osmolality of about 250 mOsm/kg, about 300 mOsm/kg, about 350 mOsm/kg, 400 mOsm/kg, about 450 mOsm/kg, about 500 mOsm/kg, about 550 mOsm/kg, about 600 mOsm/kg, and the like). This technique, combined with attempted delivery of very high concentrations of cells, represents another method of overcoming the challenges of effective cell delivery therapy.

The above reference is one example of the past misunderstanding regarding the cause of cell delivery inaccuracies. In the past it has been assumed that cell death was the cause of cell delivery problems, such as that caused by shear stress induced by a combination of time, pressure, diameter of delivery vehicle lumen, and the size of the cells being delivered. What was not realized was that cell settling in liquid solutions was an important cause of delivery inaccuracies.

Applicants have identified a cell delivery medium having characteristics designed to overcome this obstacle to effective therapy. The cell delivery medium is density matched with the cells it is delivering.

One skilled in the art recognizes that the internal diameter (I.D.) of the delivery tube affects the fluid dynamics of delivered solutions. For example, in a 0.012 inch inner diameter, 60 inch length catheter it was possible to readily deliver a 1 centipoise fluid but not a 5 centipoise fluid at the pressures used. In a similar example, in a 0.017 inch inner diameter, 12 inch length catheter it was possible to readily deliver fluids up to and including 50 centipoise. These characteristics will optimize cell viability, ease of physician delivery, and patient comfort and recovery.

It is recognized then that various internal diameters of catheters can be used with selected cell density solutions (including, but not limited to, 0.017 in., 0.016 in., 0.014 in., 0.0135 in., 0.0012 in., 0.009 in. and the like). Similarly, because of the high survivability rate demonstrated for cells in these solutions, much higher shear rates can be used than previously believed possible, including but not limited to rates equal to or greater than 1000 1/sec, 2000 1/sec, 3000 1/sec, 4000 1/sec, 5000 1/sec, 6000 1/sec, 7000 1/sec, 8000 1/sec. One feature of the described cell delivery fluids is that they permit cells to survive much higher shear stress in catheters (including but not limited to equal or greater than 1 N/m², 2 N/m², 3 N/m², 4 N/m², 5 N/m², 6 N/m² and the like). One skilled in the art would recognize that the survivability of cells is proportional to the shear stress in the catheter and the length of time it experiences the effective shear forces. It is recognized that the effective time that time a cell experiences an effective shear stress in the catheter may be as short as about 10 msec to upward of 5000 msec (including ranges of less then 4000 msec, less then 3000 msec, less then 2000 msec, less then 1000 msec.) Therefore, ideal survival rates for cells may be optimized by effectively matching the delivery requirements, the shear stress, and the delivery time.

Although other catheters having varied approach for therapy delivery, namely endocardial, epicardial and intramyocardial can benefit from the inventions described here, these inventions are preferentially designed for transvenous, intramural delivery catheter, namely the TransAccess LT catheter delivery system.

It is also recognized that higher viscosities may be possible with cell delivery devices via devices of relatively shorter length and possibly of a larger lumen size, and still enjoy the benefits of this invention. Further, the present invention may use less than optimally matched cell density vehicles where the use of these vehicles with the delivered cells preferably improves at least one measurable fluid dynamic in the catheter or at least one measure of effective delivery. Consistent with the foregoing matching of vehicles is that the density of the vehicle may be within about 10%, within about 5%, within about 2%, within about 1%, within about 0.1%, or within about 0.01% of any given cell density. Known examples of media which may be appropriate, with proper formulation, include Isovue brand image enhancing media (sold by Bracco Diagnostics), perfluorooctyl bromide (perflubron), known under the Oxygent brand name (sold by Alliance Pharmaceuticals), dextran solutions, such as Dextran 40 I.P, Microspan 40 in normal saline, and MICROSPAN40 in 5% dextrose (manufactured by Leuconostoc mesenteroides).

A number of references are available for determining cell density. Listed below are some published density values for the cell types given:

Cell Specific Gravities

| | |
|---|---|
| red blood cells: | 1.10 |
| stem cells (CD34 cells): | 1.065 |
| platelets: | 1.063 |
| monocytes: | 1.068 |
| lymphocytes: | 1.077 |
| hepatocytes | 1.07-1.10 |
| granulocytes | 1.08-1.09 |

The cells delivered suspended in the described vehicles may vary widely in the actual effective cell concentration. The cell concentration may vary from about 1×10⁹ cells per milliliter to about 1×10⁸ cells per milliliter (ml) (including from about 1.7×10⁹ cells/ml, about 5×10⁸ cells/ml, about 1×10⁸ cells/ml, about 1×10⁹ cells/ml, and the like depending on cell size). Choice of the delivered concentration of cells along with the number of cells is one criteria matched in selecting the appropriate vehicle for the delivered cells and medium to the target site.

One of several goals of the vehicle of this invention is to mitigate undesired settling of the cells placed in the vehicle, if the settling of the cells becomes a cause for concern. This is done in order to achieve a known, consistent (and preferably very high) cell delivery concentration ratio, i.e., delivered cells as compared with available cells intended to be delivered by the physician to a specific site should be close to the value of 1:1. It is a similar goal to ensure that an acceptable viability ratio (preferably also near 1:1) is achieved by which a high percentage of delivered cells are functional and replicate at well accepted levels. Providing methods and compositions which achieve this goal permits vast improvement over the known delivery capabilities of this type of treatment and improves the reliability of this form of medical treatment available to millions of people.

Applicants realized through investigation that cell loss, rather than cell death, was possibly the critical issue in catheter-based cell delivery. Catheter applications would include use with cardiac delivery catheters; such as the TransAccess catheter delivery system (Medtronic, Inc,) Use of cell density balanced solutions also would be appropriate in other non-cardiac delivery methodologies, such as neurovascular, peripheral vascular, and the like, and more generally to syringe delivery of cells. The phenomenon of cell death observed in the first couple of weeks post-delivery may be directly related to the conditions linked to the underlying pathology, namely inflammation, ischemia, hypoxia, anoxia, degeneration of myocardial matrix, etc. The acute cell loss during delivery, may be due to false targeting, drainage into venous and/or lymphatic systems, aspects that are prevented in our invention.

In other embodiments of the invention, the bioagent comprises anti-inflammatory compounds, anti-proliferative compounds, anti-bacterial compounds, pro-cell survival compounds, analgesic compounds, nucleotide sequences, or any combination thereof.

Suitable anti-inflammatory compounds include the compounds of both steroidal and non-steroidal structures. Suitable non-limiting examples of steroidal anti-inflammatory compounds are corticosteroids such as hydrocortisone, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone. Mixtures of the above steroidal anti-inflammatory compounds can also be used.

Non-limiting examples of non-steroidal anti-inflammatory compounds include the oxicams, such as piroxicam, isoxicam, tenoxicam, sudoxicam, and CP-14, 304; the salicylates, such as aspirin, disalcid, benorylate, trilisate, safapryn, solprin, diflunisal, and fendosal; the acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; the fenamates, such as mefenamic, meclofenamic, flufenamic, niflumic, and tolfenamic acids; the propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; and the pyrazoles, such as phenylbutazone, oxyphenbutazone, feprazone, azapropazone, and trimethazone.

The variety of compounds encompassed by this group are well-known to those skilled in the art. For detailed disclosure of the chemical structure, synthesis, side effects, etc. of non-steroidal anti-inflammatory compounds, reference may be had to standard texts, including Anti-inflammatory and Anti-Rheumatic Drugs, K. D. Rainsford, Vol. I-III, CRC Press, Boca Raton, (1985), and Anti-inflammatory Agents, Chemistry and Pharmacology 1, R. A. Scherrer, et al., Academic Press, New York (1974), each incorporated herein by reference.

Mixtures of these non-steroidal anti-inflammatory compounds may also be employed, as well as the pharmacologically acceptable salts and esters of these compounds.

Generally, anti-inflammatory non-steroid drugs are included in the definition of “analgesics” because they provide pain relief. However, in this disclosure, anti-inflammatory non-steroid drugs are included in the definition of anti-inflammatory compounds. Accordingly, the definition of the term “analgesics” for the purposes of the current disclosure does not include anti-inflammatory compounds. Thus, suitable analgesics include other types of compounds, such as, for example, opioids (such as, for example, morphine and naloxone), local anaesthetics (such as, for example, lidocaine), glutamate receptor antagonists, α-adrenoreceptor agonists, adenosine, canabinoids, cholinergic and GABA receptors agonists, and different neuropeptides. A detailed discussion of different analgesics is provided in Sawynok et al., (2003) Pharmacological Reviews, 55:1-20, the content of which is incorporated herein by reference.

Suitable pro-cell survival agents include, without limitation, caspase inhibitors, non-toxic seleno-organic free radical scavengers, estrogen steroid hormones (e.g., 17-β-estradiol, estrone) and structurally related derivative compounds, and any combination thereof.

Suitable non-limiting examples of anti-proliferative agents include enoxaprin, angiopeptin, colchicine, hirudin, paclitaxel, paclitaxel analogues, paclitaxel derivatives, amlodipine, doxazosinand, and any combinations thereof.

Suitable nucleotide sequences include, without limitation, any DNA and RNA sequences capable of altering phenotypes of cells upon entry into these cells. In one embodiment, such extraneous genetic material includes, for example, siRNAs or coding sequences of genes of interest under direction of promoters, which induce expression of the coding sequences. In another embodiment, the extraneous genetic material includes sequences capable of recombination with genomic sequences thus removing selected sequences from cellular genome, which leads to decrease of expression of these removed selected sequences. Another embodiment may include the catheter-based delivery of genetically-modified cells. The protocol for delivery of genetically modified cells would be substantially the same as for non-modified cells.

A person of ordinary skill will also appreciate that the nucleotide sequences may be advantageously formulated in order to increase the efficiency of their entry into the cells. One non-limiting example of such formulation is a liposome-based formulation. Suitable techniques of preparations of such formulations are reviewed in, for example, Sambrook and Russel, Molecular Cloning: A Laboratory Manual (3^{rd} Edition), Cold Spring Harbor Press, NY, 2000, incorporated herein by reference.

A person skilled in the art will undoubtedly appreciate that if the bioagent is a molecule, rather than the cells, the bioagent may be present in the suspension in a sustained-release formulation, such as, for example, microspheres. Many methods of preparation of a sustained-release formulation are known in the art and are disclosed in Remington's Pharmaceutical Sciences (18th ed.; Mack Publishing Company, Eaton, Pa., 1990), incorporated herein by reference.

Generally, the at least one additive can be entrapped in semipermeable matrices of solid hydrophobic polymers. The matrices can be shaped into films or microcapsules. Examples of such matrices include, but are not limited to, polyesters, copolymers of L-glutamic acid and gamma ethyl-L-glutamate (Sidman et al. (1983) Biopolymers 22:547-556), polylactides (U.S. Pat. No. 3,773,919 and EP 58,481), polylactate polyglycolate (PLGA) such as polylactide-co-glycolide (see, for example, U.S. Pat. Nos. 4,767,628 and 5,654,008), hydrogels (see, for example, Langer et al. (1981) J. Biomed. Mater. Res. 15:167-277; Langer (1982) Chem. Tech. 12:98-105), non-degradable ethylene-vinyl acetate (e.g. ethylene vinyl acetate disks and poly(ethylene-co-vinyl acetate)), degradable lactic acid-glycolic acid copolymers such as the Lupron Depot™, poly-D-(−)-3-hydroxybutyric acid (EP 133,988), hyaluronic acid gels (see, for example, U.S. Pat. No. 4,636,524), alginic acid suspensions, and the like.

Suitable microcapsules can also include hydroxymethylcellulose or gelatin-microcapsules and polymethyl methacrylate microcapsules prepared by coacervation techniques or by interfacial polymerization. See the PCT publication WO 99/24061 entitled “Method for Producing Sustained-release Formulations,” wherein a protein is encapsulated in PLGA microspheres, herein incorporated by reference. In addition, microemulsions or colloidal drug delivery systems such as liposomes and albumin microspheres, may also be used. See Remington's Pharmaceutical Sciences (18^{th} ed.; Mack Publishing Company Co., Eaton, Pa., 1990). Other preferred sustained-release compositions employ a bioadhesive to retain the at least one anti-inflammatory compound and/or the additive at the site of administration.

The sustained-release formulation may comprise a biodegradable polymer, which may provide for non-immediate release. Non-limiting examples of biodegradable polymers suitable for the sustained-release formulations include poly(alpha-hydroxy acids), poly(lactide-co-glycolide) (PLGA), polylactide (PLA), polyglycolide (PG), polyethylene glycol (PEG) conjugates of poly(alpha-hydroxy acids), polyorthoesters, polyaspirins, polyphosphagenes, collagen, starch, chitosans, gelatin, alginates, dextrans, vinylpyrrolidone, polyvinyl alcohol (PVA), PVA-g-PLGA, PEGT-PBT copolymer (polyactive), methacrylates, poly(N-isopropylacrylamide), PEO-PPO-PEO (pluronics), PEO-PPO-PAA copolymers, PLGA-PEO-PLGA, or combinations thereof.

Accordingly, the methods of creating the suitable sustained-release formulations are within the expertise of the person having ordinary skill in the art.

In different embodiments of the invention, the suspension may be delivered to the targeted area via a catheter and injected to the targeted area through a wall of a blood vessel adjacent to the targeted area. For example, targeting the myocardial tissue, via the TransAccess percutaneous transvenous catheter, cells can be delivered into through the wall of anterior interventricular artery and into the anterior wall of the LV. Other regions such as lateral and posterior myocardium can also be targeted with the mentioned device and use the methods described in this invention. Non-limiting examples of suitable catheters include the TransAccess catheter delivery system. In one embodiment, the suitable catheter is Pioneer CX delivery catheter (Medtronic, Inc., Minneapolis, Minn.). In another embodiment, the catheter is a minimally invasive transvenous catheter, such as, for example, TransAccess LT (available from Medtronic, Inc., Minneapolis, Minn.).

The methods of introducing the catheter into the blood vessels are known to persons of ordinary skill in the art. In one non-limiting example, the catheter can be introduced into a femoral vein and advanced into the vessel adjacent to the targeted area.

For example, if the vessel adjacent to the targeted area is the anterior interventricular artery, the catheter may be advanced from the femoral vein through the right ventricle to the coronary sinus and then to the great cardiac vein. The catheter then penetrates the great cardiac vein and reaches the anterior interventricular artery. This procedure is described in details in the examples of the current disclosure.

A person of ordinary skill in the art will appreciate that the suspension may be delivered to the targeted area in more than one injection. An important consideration for the practitioner of the current invention is the suspension volume to be injected at a time. Another important consideration is the total volume of the suspension which can be injected safely and efficiently. The present invention provides important novel information for both of these considerations.

Applicants have found that the volume of a single injection that optimizes a retention of the bioagent in the targeted area while minimizing systemic distribution in the subject is between about 10 μl and about 200 μl, preferably, between 10 μl and 160 μl, more preferably, between 10 μl and 80 μl. If the practitioner chooses to dispense the suspension in more than one injection, the distance between the injections is, in one embodiment, at least about 2 mm, or more preferably, at least about 2.5 mm, or even more preferably, at least about 3 mm.

Applicants further provide information that the total volume of the suspension delivered to the targeted area preferably should be approximately equal to a product of the total volume of the targeted area and the interstitial capacity of the targeted area. In one embodiment, the organ is heart and the interstitial capacity is between about 0.08 ml/g and about 0.43 ml/g. In one embodiment, the targeted area is a chronic, noncalcified ischemic lesion, and the interstitial capacity of the targeted area is between about 0.12 ml/g and about 0.20 ml/g.

If the practitioner selects the injection of the suspension into the targeted area through the patient's anterior interventricular artery, the methods of the present invention provide for about 21% of the cells retained in the heart, and about 95% of these cells retained in the infarcted targeted area.

In another aspect, the current invention provides a kit for delivering a bioagent to a targeted area of an organ. The kit provides a delivery device, a contrast agent, and a vehicle, as described above in this disclosure.

In one embodiment, the kit further comprises a bioagent to be introduced into the targeted area of the organ in accordance with the methods provided by this invention. According to one embodiment of the current invention, the bioagent may comprise cells, proteins, drugs, nucleic acids, or a combination thereof. The cells may be selected from the group consisting of myoblasts, embryonic stem cells, adult stem cells, and any combination thereof, and derived from brain, bone marrow, peripheral blood, cord blood, blood vessels, skeletal muscle, skin liver, and heart. In one embodiment, the cells are received by the user at concentrations up to about 170×10⁷/mL, e.g., up to about 170×10⁶/mL, up to about 170×10⁵/mL, or up to about 100×10⁵/mL.

At least a portion of the cells included with the kit may be labeled with a marker, such as, for example, europium nanoparticles and/or superparamagnetic iron oxide particles.

In another embodiment, the marker is provided independently of the cells. If this embodiment is selected, at least the portion of the cells may be labeled with the marker, such as, for example, europium or any other marker described above, at the time of the practitioner's choice.

An alternative way for incorporating Isovue and other contrast agents has been developed. A practical and efficient method includes the use of an increased cell concentration which is prepared prior to cell delivery, and mixed with commercially available Isovue to generate a mixture that is cell friendly, fluoroscopically visible and amenable for delivery via the TransAccess catheter. The kit may further comprise a set of instructions. The set of instructions preferably includes information necessary for proper use of the kit, such as, for example, instructions on handling and labeling the cells with the marker, instructions on mixing the contrast agent, the vehicle, and the bioagent, instructions on delivering the suspension, whether by direct injection or by injection into the blood vessel supplying blood to the targeted area and other instructions necessary or desirable to provide the practitioner to be able to use the kit of the present invention safely and efficiently.

A person of ordinary skill in the art will appreciate that the set of instructions can be in any suitable medium, including, without limitation, printed, video-taped, digital, and audio-recorded.

Specific embodiments according to the methods of the present invention will now be described in the following non-limiting examples.

### EXAMPLES

### Example 1

### Fibroblast Suspensions do not Maintain their Initial Concentration when Allowed to Sit Over Time

Fibroblast cells were stored in 50 mL centrifuge tubes over a period of 100 minutes, both on ice and at room temperature (RT). Samples were removed by pipette from the top and bottom of both the ice and RT suspensions every 20 minutes. No mixing was done for the first 60 minutes. At the 80 minute time point, gentle mixing (hand swirling) was done immediately before sampling. At the 100 minute time, hard mixing (vigorous hand swirling) was done immediately before sampling.

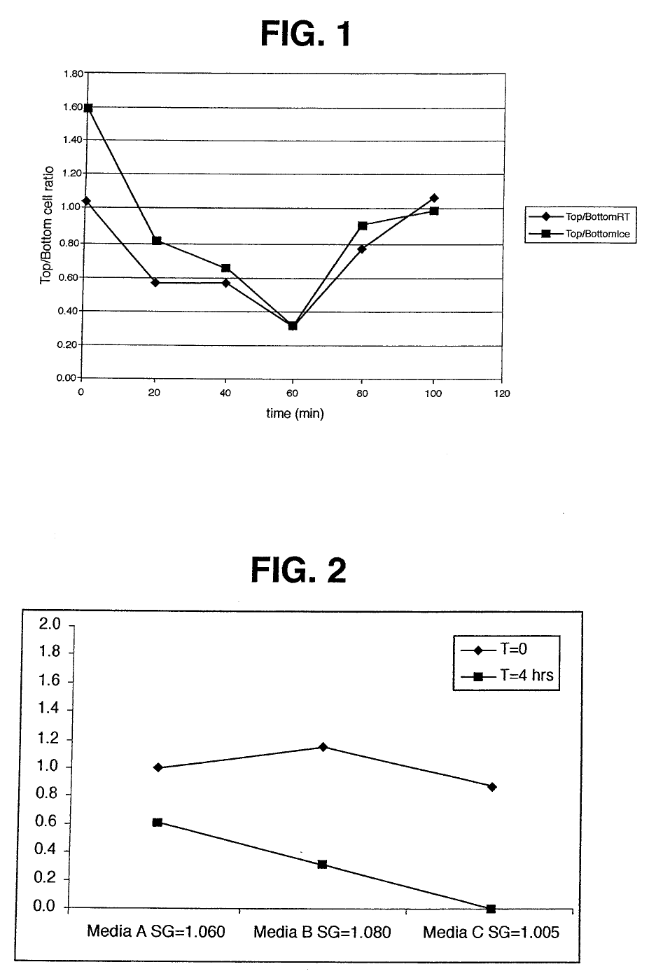

FIG. 1 illustrates the top/bottom cell count ratios as the results of this experiment. The stratification of a static fibroblast suspension, whether kept at room temperature or on ice, is clearly demonstrated. By 60 minutes, the cell concentration taken from the top of the suspension was only 30% of that taken from the bottom. But after a gentle mix (the 80 minute time point), suspension equilibrium was clearly restored.

The results shown in Example 7 demonstrate that fibroblast suspensions do not maintain their initial concentration when allowed to sit over time. The results further suggest that settling of the suspensions is occurring, but that even gentle mixing brings these suspensions back into equilibrium. This finding has potential impact on delivery device, delivery medium, and overall delivery system design, as it will be critical to assure that the appropriate concentration of therapeutic cells can be delivered through the catheter repeatedly and reliably. However, recognizing that rapid settling may occur, then constant agitation of a delivery vehicle and injectate medium may be necessary to prevent such phenomenon. But as a practical matter such agitation is not desirable by the physician. Consequently, Applicants identified a new solution to achieve a matched density of the vehicle with the cells being delivered by that vehicle.

Prevention of human fibroblast settling was investigated using isotonic diluted solutions of Isovue brand image enhancing media to increase the specific gravity of the cell media above normal saline.

### Example 2

### High Density Solutions Significantly Slowed the Fibroblast Settling

A cell delivery medium was density matched with cells as a dilution of Isovue-300 image enhancing media (sold by Bracco Diagnostics) and human dermal fibroblasts. Isovue is a non-ionic image enhancing media with the active agent of iopamidol. The package insert for Isovue-300 lists the concentration as 300 mg/mL (61%), osmolality of 616 mOsm/kg water, viscosity at 20 C as 8.8 cP, and specific gravity of 1.339. Isovue-300 was then diluted 1:2 v/v (1 part Isovue to 1 part deionized water). The 1:2 diluted Isovue osmolality is about 300 mOsm/kg, and the calculated specific gravity is 1.170. The fibroblasts suspended in Hanks Balanced Salt Solution (HBSS) were then diluted with the diluted Isovue media to achieve a specific gravity of 1.060. Since the osmolality of both HBSS and diluted Isovue media is about 300 mOsm/kg, the dilution does not change the osmolality of the diluted cell suspensions.

The specific gravities of the solutions tested were 1.060 (Media A), 1.080 (Media B), and 1.005 (control-Media C). The cell concentrations on the top and bottom of the three solutions were counted before and after 4 hours of settling time. As shown in FIG. 2, both the high density solutions significantly slowed the fibroblasts settling compared to the control. A comparison of top layers over time can also be made. After 4 hours, the control had zero cells in the top layer, and the diluted Isovue solutions had 105 & 57% of the initial cell count in the top layer. After 4 hours, the bottom layer for all the solutions contained more cells than counted initially.

Trypan cell counts (using Trypan Blue solution from Sigma Chemical) were performed at the two time points (0 & 4 hours). The number of stained (dead) cells were not significantly different for the diluted Isovue solutions compared to the saline control. Diluted Isovue media did not appear to significantly rupture cell membranes after 4 hours of contact. The cell proliferation assay indicated the fibroblasts proliferated after exposure to Isovue as well as with the saline control.

### Example 3

### High Density Solutions Significantly Slowed the Myoblast Settling

This experiment was very similar to that performed in Example 2, except that myoblasts were used rather than fibroblasts. Prevention of myoblast settling was investigated using isotonic diluted solutions of Isovue image enhancing media to increase the specific gravity of the cell media above normal saline. The specific gravities of the solutions tested were 1.060 (Media A), 1.080 (Media B), and 1.005 (control-Media C). The cell concentrations on the top and bottom of the three solutions were counted before and after 4 hours of settling time.

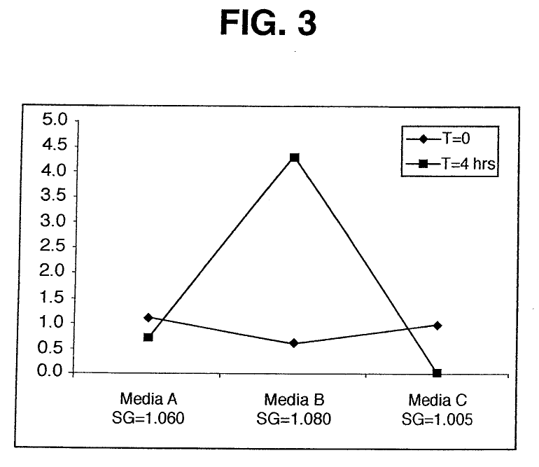

As shown in FIG. 3, both the high-density solutions significantly slowed the myoblasts settling compared to the HBSS control. After 4 hours, the control had zero cells in the top layer, and the diluted Isovue solutions had 75 & 85% of the initial cell count in the top layer. After 4 hours, media A& B had strands of cells suspended with the control having some of the cells clumped on the bottom layer of the centrifuge tube. Strands of myoblasts after four hours were unexpected, as this was not observed with fibroblasts. The centrifuge tubes were gently mixed by hand swirling prior to the proliferation assay. These cell counts, after gentle mixing, indicate 24-40% of the cells were lost after four hours due to adherence to the vessel wall or each other (clumping). Cell settling was a more significant issue than adherence as the control had a 29-fold increase of cells on the bottom of the tube after four hours of settling. The density of the myoblasts is approximately 1.06 g/mL. The number of Trypan stained (dead) cells were very few and not significantly different for the three solutions. Diluted Isovue media does not appear to significantly rupture the myoblast cell membranes after 4 hours of contact. The cell proliferation assay indicates the myoblasts proliferated as well after exposure to Isovue as prior to exposure.

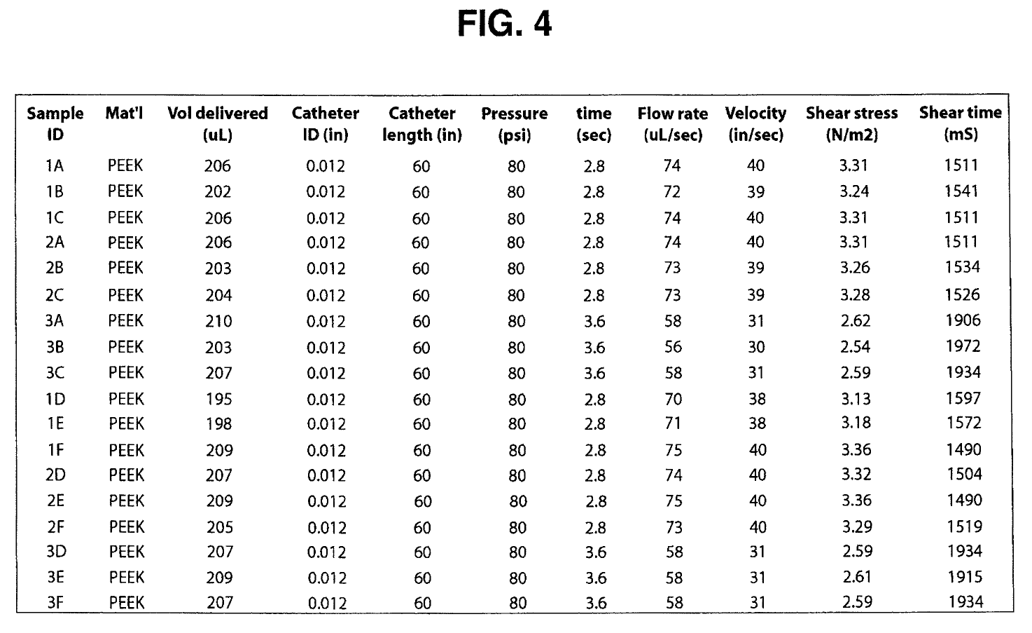

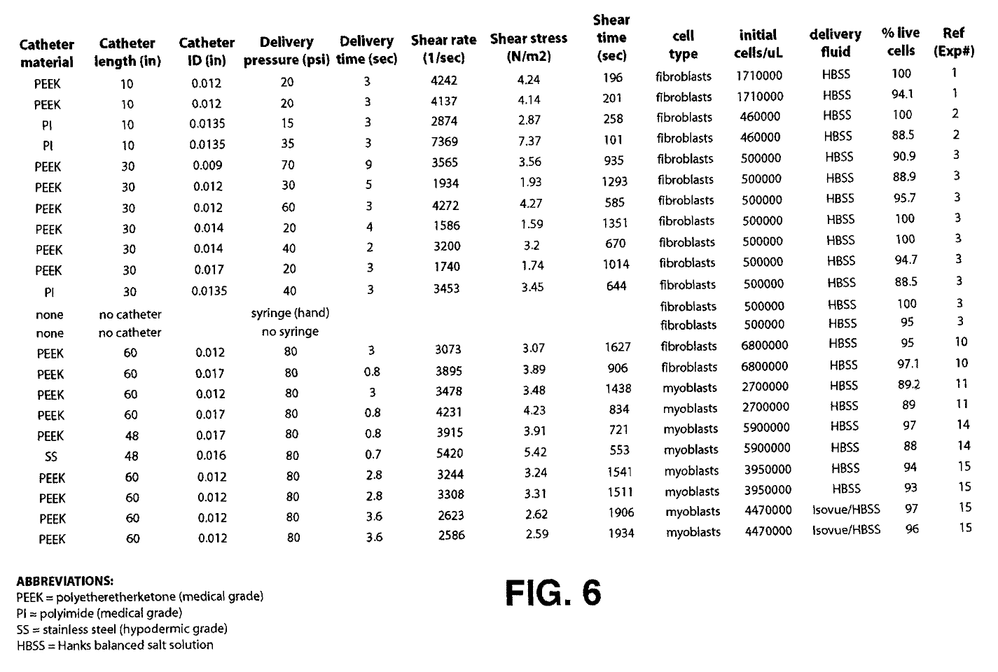

Applicants' previous experiments demonstrated that by matching the density and osmolality of a vehicle to that of the cells being delivered, then settling of cells can be minimized. Further experiments were done in view of the known problem of possible damage to cells due to adhesion with various materials. Although Applicants successfully performed experiments which verified that viable, proliferative myoblasts can be delivered consistently through a wide range of catheter materials. Catheter materials may include various polymers, including but not limited to, poly etheretherketone (PEEK), polyimide (PI—medical grade), polyurethanes, polyamides, silicones, polyethylenes, polyurethane blends, polyether block amides (e.g., PEBAX), and the like, or including various metal materials, including but not limited to stainless steel (SS), titanium alloys, nickel titanium alloys (e.g. Nitinol), chromium alloys (MP35N, Elgiloy, Phynox, etc.), cobalt alloys, and the like. More preferably the catheter materials are chosen from the group of poly etheretherketone (PEEK), polyimide (PI), and stainless steel (SS). One feature studied when cells were delivered through catheters (after proper mixing in the cell density solution) was the behavior of myoblasts and fibroblasts in the various vessels used to hold, transport, and inject cells over the expected implant period. Accordingly, further experiments included combinations of density matched and non-density matched delivery media in catheters of lengths, lumen sizes, and materials representative of those suitable for transvenous cell delivery. As shown in FIGS. 4 and 6, and used in the investigations of Examples IV and VI below, some of the media experience relatively high shear time, which was previously believed to be a key indicator of adhesion and cell delivery inaccuracies.

Example 4 evaluates the two technologies of cell delivery and prevention of cell settling performed simultaneously by delivering myoblasts through catheters using cell settling prevention media. The investigation focuses on the variation of parameters and their effect on cell survival. The design parameters of interest include pressure, flow rate, catheter diameter, catheter length, and cell concentration. Concentrations and survival rates of cells delivered from the settling prevention media are measured and compared to those of cells delivered from HBSS. Cells are allowed to settle for 40 minutes in an effort to determine whether cells suspended in settling prevention media can be delivered without the need for mixing.

### Example 4

### The Use of a Cell Settling Prevention Media Allows For Delivery of the Initial Concentration of Cells

Three separate delivery solutions were prepared. The cell concentration was held constant by using the ratio (2 mL cell suspension/1 mL vehicle solution) for a total of 3 mL added to each of three delivery syringes. The cell suspension was the same in all three solutions. The makeup of each of the three vehicle solutions was as follows:

Solutions 1 and 2: Hanks balanced salt solution (HBSS) as used in previous experiments;

Solution 3: Isovue 370/deionized (DI) H₂O mix, adjusted for a specific gravity of 1.060 and an osmolality of 300 mOsm/kg.

Solution 3 was prepared similarly to the cell settling prevention media of previous experiments, with the exception that Isovue 370 image enhancing media was used in place of Isovue 300. Isovue 370 is simply a more concentrated iopamidol solution than Isovue 300, and it was found that an additional dilution with DI H₂O (3.8 mL of DI H₂O for every 16.2 mL of Isovue 370) brought the properties of Isovue 370 media back to those of Isovue 300 media. Dilutions then continued as per the above referenced method in Example 3.

Cells and vehicle solutions were mixed in 50 mL centrifuge tubes, labeled Solutions 1, 2, and 3, and used as described below. Catheter assemblies 152.4 cm (60 inches) long were built from 0.012″ ID PEEK tubing. Myoblasts were stored in 50 mL centrifuge tubes throughout the experiment and were never frozen before use. Cells were delivered through catheter assemblies by use of the EFD Model 1500XL fluid delivery system. For each experiment, the following data were collected:
Hemocytometer counts, and Trypan Blue viability staining, on myoblasts from each of the three initial 50 mL centrifuge tubes;

Hemocytometer counts, and Trypan Blue viability staining, on myoblasts from each solution immediately after the (t=0 minutes) delivery through their respective catheters; and
Hemocytometer counts, and Trypan Blue viability staining, on myoblasts from each solution immediately after the (t=40 minutes) delivery through their respective catheters.

Tables 1, 2, and 3 below show the mixing protocols for each solution. Briefly, Solution 1 is the HBSS/cells solution, mixed before t=0 but not before t=40; Solution 2 is the HBSS/cells solution, mixed before both t=0 and t=40; and Solution 3 is the Isovue/cells solution, mixed before t=0 but not before t=40.

TABLE 1
Solution #1: HBSS, no mix at 40 min
(t = 0: 11:35 am; t = 40: 12:15 pm)

1A (t = 0): 4.30 M, 95% viable
1B (t = 0): 3.80 M, 94% viable
1C (t = 0): 4.30 M, 94% viable
1D (t = 40): 1.93 M, 92% viable
1E (t = 40): 1.43 M, 97% viable
1F (t = 40): 1.33 M, 95% viable

TABLE 2
Solution #2: HBSS, mix at 40 min
(t = 0: 11:48 am; t = 40: 12:28 pm)

2A (t = 0): 4.20 M, 93% viable
2B (t = 0): 3.83 M, 93% viable
2C (t = 0): 3.83 M, 93% viable
2D (t = 40): 4.10 M, 91% viable
2E (t = 40): 3.98 M, 91% viable
2F (t = 40): 3.93 M, 88% viable

TABLE 3
Solution #3: Isovue/HBSS, no mix at 40 min
(t = 0: 12:01 pm; t = 40: 12:41 pm)

3A (t = 0): 4.18 M, 97% viable
3B (t = 0): 4.03 M, 96% viable
3C (t = 0): 4.25 M, 96% viable
3D (t = 40): 3.75 M, 96% viable
3E (t = 40): 3.93 M, 93% viable
3F (t = 40): 4.00 M, 94% viable

Canine skeletal myoblasts were cultured until sufficient cells were available. The myoblasts were dissociated, rinsed, and re-suspended in HBSS into a 50 mL centrifuge tube containing the appropriate vehicle solution. In this experiment Applicants were able to deliver a minimum of 1 million cells/mL into the catheters.

Cells from all samples described in the preceding sections were manually counted in duplicate using a hemocytometer. Table 4 shows the cell count ratios, with units in cells/mL.

TABLE 4

| | | | t = 0 delivered | t = 40 delivered | Ratio, | Ratio, |
|---|---|---|---|---|---|---|
| Solution | Delivery | Initial cell | cell concentration | cell concentration | t = 0/initial | t = 40/t = 0 |
| # | solution | concentration | (average) | (average) | (% of init) | (% of t = 0) |
| 1 | HBSS, no mix | 3.95 M | 4.13 M | 1.56 M | 104% | 38% |
| 2 | HBSS, mix | 3.95 M | 3.95 M | 4.00 M | 100% | 101% |
| 3 | Isovue, no mix | 4.47 M | 4.15 M | 3.89 M | 93% | 94% |

The results of Example 4 clearly demonstrate that use of a cell settling prevention media (in this case, a dilute solution of Isovue 370 media) allows for delivery of the initial concentration of myoblasts, even after 40 minutes without mixing have elapsed. The myoblast concentration after delivery from the settling prevention media is essentially unchanged after 40 minutes (94% of t=0 concentration), whereas significant numbers of myoblasts are lost after delivery following 40 minutes in HBSS without mixing (only 38% of the t=0 concentration was delivered). These results clearly show the effectiveness of cell settling prevention media for retaining myoblast concentrations in catheter delivery, even without mixing.

### Example 5

### Effect of Specific Gravity Matched Solutions

Human dermal fibroblasts were harvested, counted, and equally divided into two separate tubes. The number of cells in each tube was approximately 375 million cells. The makeup of each tube was as follows:

Solution “−” Hanks balanced salt solution (HBSS) with cells

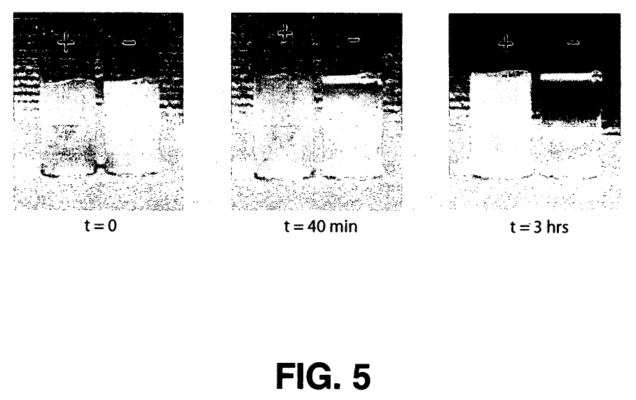

Solution “+”: Hanks balanced salt solution with cells mixed with Isovue 370, adjusted for a specific gravity of 1.060 and an osmolality of 300 mOsm/kg. The tubes were left at room temperature and pictures were taken at times 0.40 minutes, and 3 hours (FIG. 5). FIG. 5 illustrates the effect that specific gravity matched solutions with Isovue 370 have, compared to normal Hanks balanced salt solutions on cell settling.

### Example 6

### Performance of Cell Delivery Fluids Across Different Catheter Systems

Several tests were made to evaluate the performance of cell delivery fluids across different catheter systems varying the materials, lengths, diameters, and delivery pressures (see also general catheter assemblies below) for different cell types (e.g., fibroblasts and myoblasts—see also cell preparation below) (FIG. 6). Based on the various delivery parameters, e.g., shear rate, shear stress, shear time (see also fluid flow parameters below), percent of live cells resulting from delivery was measured.

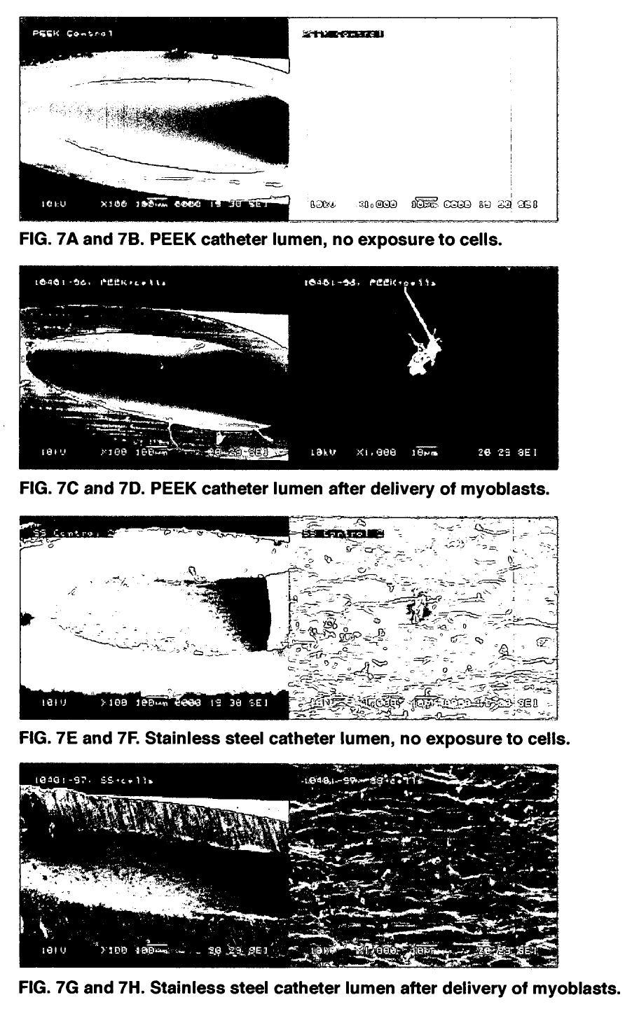

FIGS. 7A through 7H are SEM photographs of lumenal catheter surfaces. In each pair of figures, the image on the left was taken at 100× magnification, and the image on the right at 1000×. The 1000× images are representative areas from the approximate centers of the analogous 100× images: FIGS. 7A and 7B are photographs of PEEK catheter lumens after no exposure to cells; FIGS. 7C and 7D are photographs of PEEK catheter lumens after delivery of myoblasts; FIGS. 7E and 7F are photographs of stainless steel catheter lumens after no exposure to cells; FIGS. 7G and 7H are photographs of stainless steel catheter lumens after delivery of myoblasts. These images indicate that the cell delivery vehicles left very few residual cells on the internal surfaces of the catheter.

General Cell Preparation

Human dermal fibroblasts, (Clonetics, Inc.) or, in later experiments, canine skeletal myoblasts, were cultured in tissue culture flasks using specialty growth media (Clonetics, Inc.). The media was replaced every three days and when confluent, the cells were passaged to propagate the cultures. After it was determined that sufficient cells were available, the cells were rinsed once with Hanks balanced salt solution (HBSS) and then dissociated with a 5 min enzymatic wash (0.25% trypsin) at 37° C. The resulting cell suspension was neutralized with serum containing growth media and then centrifuged (800 g) for 10 min to pellet the cells. The supernatant was discarded and the pellet was resuspended in HBSS solution. At this point, the approximate cell concentration was determined by a hemocytometer cell count. The volume of HBSS was adjusted to obtain the desired cell concentration. The initial cell concentration, was calculated from the hemocytometer cell count and HBSS dilution. The final cell suspension was stored under ice for the duration of the experiment.

General Test Catheter Assembly:

The test catheter assemblies were made by bonding segments of PEEK (polyetherether ketone), PI (polyimide), [or in later experiments, stainless steel (SS)] of various lengths and diameters to luer-lock stub adaptors with Loctite 401 adhesive after priming with Loctite 7701.

General Fluid Flow Set-Up:

The fluid flow setup consisted of a fluid dispenser (EFD, Model 1500XL) driven by compressed air (max 85 psi) fitted with a 3 cc syringe. The fluid to be dispensed (either the cell suspension of interest or DI water) was loaded into the syringe. The syringe tip was fitted with the test catheter assembly described in the previous section. Delivery time (to the nearest 0.1 second) and pressure (up to 80 psi) can be fixed with this system. To ensure that a suitable volume of cell suspension was delivered, preliminary flow rate measurements were done with DI water.

### Example 7

### Cell Preparation and Labeling

Cells

Allogenic porcine skeletal muscle myoblasts were scaled up at Genzyme Corp. (Cambridge, Mass.) for four weeks. The day before harvesting, the cells were labeled with europium and iron nanoparticles using a liposome delivery method described below. Following harvesting, the cells were shipped, overnight, to Medtronic and arrived one day prior to the injection procedure. A minimum of 800×10⁶ cells were requested for each animal of which 100×10⁶ were used for making a standard curve for the europium analysis. The remaining cells were used for the delivery procedure.

Cell Preparation

Approximately 20 minutes prior to injection, the cells were pelleted using 210 to 310 RCFs for 5 minutes with slow deceleration. The supernatant was discarded and the cell pellet was resuspended in injectate buffer. The injectate buffer consisted of approximately 30% Isovue-370 (v/v), 0.3% Toluidine-blue dye (w/v) in distilled water with an osmolality of 290±5 mmol/kg. The injectate buffer was made with a new bottle of Isovue in each case and within 24 hours of its use. The final volume of the resulting cell solution available for injection was within the range of 5.6 to 6 mL.

Cell Labeling

The cell scale up and labeling was performed at Genzyme's Sydney Street facility. The labeling procedure consisted of replacing the normal growth media with media containing Europium/liposome complexes (labeling media) the day before harvesting the cells. The labeling procedure is as follows:
1. To a sterile 250 mL plastic bottle add 12 mL of lipofectamine and 12 mL of Europium stock solution to 200 mL of PMM growth media.
2. Place the resulting solution on a rocker platform at room temp with gentle agitation for 30 minutes.
3. Add the lipofectamine-europium solution to 1800 mL of PMM growth media.
4. Gently mix and add to the resulting 2 L of “labeling media” to the Cell Factory (100 mL/layer could be less if more than 20 layers were required).
5. Incubate overnight in a cell culture incubator to allow cells to uptake the Eu/liposome complexes.
6. Remove the transfection media and rinse the cells with fresh growth media. Harvest the cells with trypsin. It is necessary to note that the transfection procedure tends to weaken the cells adherence properties slightly this is why growth media is used to rinse the cells rather than HBSS.
7. Run viability tests and set up differentiation assays prior to shipment.
8. Count and resuspend the cells in the proper cell shipping media.

### Example 8

### Delivery of the Suspension to the Targeted Area

Cells

The cells were prepared as in Example 7.

Direct Injections

Since the injection protocol includes delivery of 30 injections (200 μL each) and the infarcts following LAD ischemia typically compromised septum and LV free wall (LVFW), ⅓ of the injections (10) were delivered to the septum and ⅔ into the LVFW. The direct injections in the anterior LVFW and the apex were done by inserting the bent needle parallel to the surface of the myocardium, approximately 2-3 mm deep and 5 mm lengthwise. The needle was then drawn back approximately 1 to 2 mm to create a channel in the myocardium before injecting 0.2 mL of the cell solution. After a few seconds the needle was advanced approximately 5 mm and the second 0.2 mL bolus was delivered in the same fashion. For septal injections the needle was inserted perpendicular to the myocardium to a depth of approximately 10 to 17 mm. It is important to note that these animals were not on cardiopulmonary bypass.

Catheter Delivery and Injections

The femoral artery and vein were isolated and cannulated. A 6 F pigtail catheter was advanced into the LV, and ventriculograms were captured. A 7 F AL1.0 guide catheter was advanced to the aortic sinus, left coronary angiograms were captured to visualize the LAD anatomy. Coronary Sinus (CS) access was gained with a Cook 7 F SIM1 diagnostic catheter. The 10.5 F CSO was then advanced over the diagnostic catheter, with a 0.038 guide wire inside of the diagnostic catheter for support, and into the CS. The diagnostic catheter and wire were removed. A Cougar 0.014″ guide wire was advanced into the Gread Cardiac Vein (GCV). A Swan Ganz catheter was advanced over the wire and into the GCV in order to capture a venogram road map. The Swan Ganz catheter was removed, and the 0.014″ wire was deep seated into the Anterior Interventricular artery (AIV). The LT catheter was advanced over the wire and guided into the AIV. A 1 cc syringe containing cell solution was attached to the Intralume delivery catheter's proximal end to prime the catheter. The Intralume catheter was then inserted into the LT catheter and advanced to the needle housing.

The 3 week post infarct MRI data was evaluated before each delivery procedure to determine the location and size of the infarct. Consistent with the direct injection group, in each of the four catheter delivery cases the cell injections were divided between the interventricular septum and the left ventricular anterior Left Ventricle Free Wall (LVFW). One third (approximately 10 injections) were delivered to the septum and two thirds (approximately 20 injections) were delivered to the LVFW. A total of 200 μL of cell solution was delivered during each individual injection. IVUS information was used to guide the rotation of the LT catheter to a position in which needle deployment would accurately target myocardium and avoid major vessels (i.e. the LAD). Fluoroscopy was used to track the position of the platinum ring located at the Intalume's distal end. Tactile feedback along with fluoroscopy and IVUS were used to determine appropriate targeting. Several observations were recorded during each injection, these included: injection No., syringe No., Intralume tract No., targeted tissue, position of LAD on IVUS, LT needle setting, resistance felt during Intralume advancement (score of 0 to 5), Intralume extension, volume delivered, if a contrast cloud formed in the tissue, and if an ectopic beat was observed. IVUS images were collected immediately after each needle deployment and fluoroscopy images were collected at the end of each tract while the Intralume was still extended.

Post Cell Injection MRI

A post cell injection MRI was performed within 1 to 2 hours of the last injection. Delayed enhancement cMRI scans using the gadolinium contrast agent, Omniscan™ (Amersham), were used to visualize the infarct. Infarcted tissue appeared more hyper intense in the MRI image using this technique. The iron nanoparticles inside the transplanted myoblasts appeared as very distinct hypo intense spots.

The short axis scans for each animal were compiled and analyzed using the 3D reconstruction software, 3D-Doctor by Able Software Corp. (the free Internet demo version). The 3D reconstruction was created by manually tracing the outline of the infarct, cells, epicardial surface and endocardial surface. The software then assigned different colors to each area.

### Example 9

### Analysis of Retention and Distribution of Delivered Cells

The europium nanoparticles used to label the cells are stable until activated by neutron activation. Upon activation these stable isotopes become radioactive allowing them to be detected with great sensitivity. The general principle underlying neutron activation is that an incident neutron is captured by an atom forming a radioactive nucleus. An ideal radioactive nucleus for use as a label is short-lived and emits a gamma-ray during the decay process. The energy of the gamma-ray is discrete and distinct for each stable atom. Specialized, high-resolution detection equipment can then be used to identify and measure the emitted gamma-ray. The number of emitted gamma-rays is directly proportional to the total mass of the parent isotope, and therefore is proportional to the total concentration of labeled research product. Although there are several modalities for analysis, the short-enhanced analysis was used in this study. Table 5 summarizes the retention rate of the cells delivered to the hearts by catheter and direct injection.

TABLE 5

| | | | | Infarct size | Cells retained | Average % |
|---|---|---|---|---|---|---|
| Method of | | | Cell # | (% of LV, | in heart (% of | retained in |
| Delivery | Pig # | Date | injected | at 3 wk P-I) | total delivered) | heart |
| Catheter | 328845 | May 18, 2005 | 5.93E+08 | 14.49 | 16 | 21 +/− 6.1 |
| Catheter | 329057 | Jun. 29, 2005 | 3.51E+08 | 7.28 | 27 | |
| Catheter | 329066 | Jul. 7, 2005 | 5.36E+08 | 5.51 | 27 | |
| Catheter | 329074 | Jul. 7, 2005 | 7.46E+08 | 5.86 | 15 | |
| Direct Inj | 328778 | May 11, 2005 | 5.71E+08 | 12.44 | 24 | 14 +/− 7.3 |
| Direct Inj | 328947 | May 26, 2005 | 6.99E+08 | 12.88 | 11 | |
| Direct Inj | 329069 | Jul. 13, 2005 | 5.32E+08 | 6.38 | 7 | |
| Direct Inj | 329076 | Jul. 13, 2005 | 7.71E+08 | 2.87 | 13 | |

Table 5 summarizes each case in this study including the % of cells retained in the heart and overall average for each group.

During necropsy the lungs, liver and kidneys were explanted and weighed. Two small (10-20 gram) tissue samples were then collected from each organ and weighed. One sample was used for the Prussian blue staining and the other was sent to BioPal Inc., for europium analysis. The results shown in tables 6 and 7 were generated by normalizing the europium signal detected in tissue samples (sent to BioPal) to the total organ weight from each animal. It should be noted that this calculation was based on the assumption that uniform distribution of the cells occurs in these organs.

TABLE 6

| | Pig # | | | | |
|---|---|---|---|---|---|
| | 328845 | 329057 | 329066 | 329074 | Average |
| Left Lung | 24% | 35% | 28% | 26% | 28% |
| Right Lung | 18% | 52% | 50% | 40% | 40% |
| Left Kidney | 0% | 0% | 0% | 0% | 0% |
| Right Kidney | 0% | 0% | 0% | 0% | 0% |
| Liver | 9% | 0% | 0% | 0% | 0% |
| Total | 50% | 88% | 78% | 66% | 70% |

Table 6 describes the estimated percentage of cells retained in the lungs, liver and kidneys for the pigs in the catheter delivery are based on the total number of cells delivered. Note: these determinations are based on the experimental assumption that uniform distribution of the cells occurs in these organs.

TABLE 7

| | Pig # | | | | |
|---|---|---|---|---|---|
| | 32877845 | 3289479057 | 32907666 | 329069 | Average |
| Left Lung | 38% | 19% | 32% | 30% | 30% |
| Right Lung | 67% | 21% | 33% | 26% | 37% |
| Left Kidney | 1% | 0% | 0% | 1% | 0% |
| Right Kidney | 1% | 0% | 0% | 0% | 0% |
| Liver | 3% | 3% | 0% | 3% | 2% |
| Total | 110% | 44% | 65% | 61% | 70% |

Table 7 describes the estimated percentage of cells retained in the lungs, liver and kidneys for the pigs in the direct injection delivery arm based on the total number of cells delivered. Note that this technique is based on the assumption that uniform distribution of the cells occurs in these organs.

These results lead to several important conclusions. First, the iron component (Feridex) used in the technique for labeling myoblasts provided an effective substrate for MRI visualization of the suspension in vivo. The distribution of the Iron-positive images was variable and evidenced a partial coverage of the infarct. The 3D MRI reconstruction indicate a comparable distribution by the two delivery approaches used here, namely, catheter-based and direct needle injection.

The Europium component of the myoblast labeling technique used in this study was effective and useful to quantitatively determine the number of cells retained in the heart following 2-3 hours post-cell delivery. The determination of disintegrations per minute (dpm) reported in myocardial tissue (BioPal) correlated to 21±6.7% of cells delivered via catheter and 14±7.3% of cells delivered via direct needle injection. Although not statistically significant, these data suggest an increased delivery efficiency (based on cell retention) when using the TransAccess LT delivery catheter system for delivering cells into the myocardium.

A Europium-based quantitative assessment of infarcted tissue showed that 20±6% and 11±8% of cells initially delivered were deposited and retained in the infarcted (infarct and marginal tissue) lesions after catheter-based and direct injection respectively. These values correlated to 95% vs. 76.5% of cells retained in the heart, indicating an increased efficiency and accuracy for delivering cells into infarcted lesions when using the catheter-based approach.

No acute complications attributable to the delivery procedure, such as pericardial blood effusion or sustained arrhythmias were noted during the cell delivery procedure (either catheter-based or direct injection) or during the subsequent cMRI evaluation. Note that the animals were euthanized 2-3 hours post cell delivery, so this observation is limited to immediate perioperative.

Macroscopic observation of explanted hearts from the catheter-based group, evidenced mild perivascular subepicardial hemorrhage, typically circumscribed to vein segments (mid-distal AIV) corresponding to sites of catheter-needle deployment during cell delivery. Likewise, myocardial tissue with blue coloration (Toluidine blue) was evident through a translucent epicardium. Hearts from the direct injection group showed punctuate hemorrhages in areas of delivery, likewise, regions of blue coloration in myocardium were evident. In a couple of phases, the superficial small vasculature at sites of delivery was observed having blue coloration.

Assessment of specimens from distant organs based on europium content showed a significant biodistribution 0 delivered cells to the lungs. Thus, 70% of cells initially delivered to the heart were detected in the lungs. The rate of biodistribution into other organs was similar for both delivery approaches.

### Example 10

### Determination of Relationship Between the Efficiency of Delivery and the Injection Volume

Cells

Cells were prepared and labeled as described in Example 1, then resuspended in a suspension containing Toluidine blue dye and Isovue. The study was designed to evaluate two volume conditions: Group I, 50 μL/injection (“low volume”) and Group II, 200 μL/injection (“High volume”). A total of 1.5 mL of cell suspension was delivered in each animal of Group I, and a total of 6 mL was delivered in each animal of Group II. The intramyocardial cell delivery was conducted via direct needle injections using the 27 GA Bent Treatment Needle from Genzyme Corp.

Study Design

In normal (non-infarcted) animals, during a thoracotomy procedure, the anterior aspect of the heart was exposed. The anterior LV myocardium was explored and a 2×2.5 cm area was selected immediately lateral to the interventricular groove for intramural delivery of 20 injections into the LV free wall (LVFW). Subsequently, a 2.5 cm of the interventricular groove (close to the LVFW injections) was selected for delivery of 10 injections in the interventricular septum. A total of 30 injections was delivered in each animal, as shown in Table 8.

TABLE 8

| Group | Number of | Test Article; Myoblast | Number of | Injection | Time of |
|---|---|---|---|---|---|
| Number | Animals | Cell Suspension | Injections | Site | Euthanasia |
| I | 2 | 50 μL/injection (LOW) | 30 per heart | 1) IV Septum | Day 0 |
| II | 2 | 200 μL/injection (HIGH) | (10 in the IV septum and | 2) LV Free Wall | |
| | | | 20 in the LV FW) | | |
| | | | 0.5 cm between injections | | |

The cells were loaded into 1 cc syringes using a 10 cc master syringe and a fluid transfer device. The 1 cc syringes were equipped with a 27-gauge Bent Treatment needle (Genzyme). The cells were injected into the interventricular septum (10 injections) and the left ventricular anterior free wall (20 injections) Injections were made at volumes of either 50 μL (low volume arm) or 200 μL (high volume arm) per injection with approximately 2-3 mm depth of injection. The injections were separated by a maximum of 0.5 cm. The actual number of cells delivered in each pig is described in Table 9.

TABLE 9

| | | | # of | Cell | Total Vol | |
|---|---|---|---|---|---|---|
| | Cell lot | Delivery arm | injections | Concentration | delivered | Total cell # |
| Pig # | used | (μL/injection) | per heart | [Cells/mL] | (mL) | delivered |
| 5P161 | MDT15-4 | 200 | 30 | 1.00E+08 | 6 | 6.00E+08 |
| 5P215 | MDT19-4 | 200 | 30 | 1.26E+08 | 6 | 7.54E+08 |
| 5P214 | MDT16-4 | 50 | 30 | 1.11E+08 | 1.5 | 1.67E+08 |
| 5P216 | MDT20-4 | 50 | 30 | 1.26E+08 | 1.5 | 1.89E+08 |

In each case, the pig was termed ten minutes after the last injection. The hearts and lungs were removed, placed in sealed plastic bags and stored at 40 C until being dissected the following day. The kidneys, liver and spleen were removed and weighed individually. A representative tissue specimen from each of these organs was then removed, weighed and placed in a BioPal sample vial. The following day, the entire right and left cardiac ventricles were dissected, and specimens were placed in BioPal sample vials. A representative tissue specimen from each lung was then removed, weighed and placed in a BioPal sample vial. All of the samples were then placed in a 60° C. oven for 2 days to dehydrate after which they were shipped to BioPal Inc. for europium analysis.

The target tissue, normal myocardium retained 18.5±3.5% of the injected cells in group I (50 μL/injection×30 and 10.5±2% in group II (200 μL/injection×30). Thus, the cell injection performed at 50 μL/injection volume indicated a 76% higher cell retention rate than its counterpart delivery at 200 μL/injection protocol.

### Example 11

### Co-Labeling of Porcine Myoblasts with Europium Nanoparticles and Feridex (Iron) Nanoparticles

In vitro experiments were performed to verify that cells could be effectively co-labeled. Porcine myoblasts were successfully co-labeled in vitro using the method described below.

1. Preparation of labeling media. The following solutions were made just prior to labeling the cells.

Solution A
i. 15.4 μL of Feridex stock
ii. 52.5 μL of Lipofectamine stock
iii. 7 mL of growth media

Solution B
iv. 84 μL of Europium stock
v. 84 μL of Lipofectamine stock
vi. 14 mL of growth media

2. Four T-75 flasks of passage 4 porcine myoblasts were incubated overnight in the following set of media conditions.
i. Control (10 mL of growth media only)
ii. Feridex and Lipofectamine (6.66 mL of growth media+3.33 mL of solution A)
iii. Europium and Lipofectamine (6.66 mL of solution B+3.33 mL of growth media)

iv. Feridex, Europium and Lipofectamine (6.66 mL of solution B+3.33 mL of solution A).

TABLE 10

| Sample ID. | Europium signal (dpms) |
|---|---|
| Growth media control | 0 |
| Feridex + Liposomes | 0 |
| Eu + Liposomes | 812795.2 |
| Eu + Feridex + Liposomes | 817154.8 |

These results demonstrate that cells can successfully be co-labeled with iron and European nanoparticles using lipofection. Further, these studies demonstrate that iron nanoparticles do not interfere with quantitative neutron activation analysis.

### Example 12

### Effect of Isovue™ and Visipaque™ on Suspension Osmolarity and Cell Viability

Cells were prepared as described in Example 7.

The compositions and osmolarities of different suspensions are shown in the following table.

TABLE 11

| | Condition | Osmolality (mmol/kg) |
|---|---|---|
| with cells | (#1) 35% Isovue in water | 312 |
| with cells | (#2) SMSM + Isovue | 442 |
| with cells | (#3) SMSM | 295 |
| with cells | (#4) 100% Visipaque | 330 |
| with cells | (#5) SMSM + Visipaque | 325 |
| no cells | 100% Isovue | 897 |
| no cells | SMSM + Isovue | 489 |
| no cells | SMSM | 287 |
| no cells | 100% Visipaque | 277 |
| no cells | SMSM + Visipaque | 322 |

Table 11 describes the osmolarity of suspensions comprising different concentrations of Isovue™ and Visipaque™, with or without myoblasts. The osmolarity measurements were taken at room temperature.

Since the suspension comprising Visipaque™ was within an acceptable osmolarity range, the viability of the cells in the suspensions comprising different concentrations of Isovue™ and Visipaque™ was tested for up to three hours at 4° C., 20° C., and 37° C. The suspensions tested comprised Suspension 1 (myoblasts having final concentration of about 133 million/ml+35% Isovue+water), Suspension 2 (myoblasts having final concentration of about 133 million/ml+100% Isovue+SMSM), Suspension 3 (myoblasts having final concentration of about 133 million/ml+100% SMSM), and Suspension 4 (myoblasts having final concentration of about 133 million/ml+100% Visipaque+SMSM).

At 4° C. and at 20° C., the viability of the cells was approximately the same in all four suspensions (95%-100%) and did not change in three hours. At 37° C., the viability of the cells in Suspensions 1, 2, and 4 (the suspensions having a contrast agent) decreased to a larger degree than the viability of the cells in Suspension 3 (no contrast agent). After 1 and 2 hours, Suspension 3 showed 95%-100% viability and after 3 hours, Suspension 3 showed about 95% viability. The viabilities of the cells in Suspensions 1, 2, and 4 were about the same at identical time points (about 95% at 1 and 2 hours and about 85%-90% at 3 hours).

These results show that both Isovue™ and Visipaque™ do not have different effects on cell viability within the tested conditions.

An acute catheter delivery procedure (in a healthy pig) of allogenic myoblasts resuspended in different contrast formulations. The conditions tested were:
1. myoblasts formulated to 166 million/ml with SMSM, then diluted to 133 million/mL with 100% stock Isovue-370 and
2. myoblasts formulated to 166 million/ml with SMSM, then diluted to 133 million/mL with 100% stock Visipaque-320.

Both conditions produced very evident clouds under fluoroscopy during injection. Therefore, both iodixanol (Visipaque™) and iopamidol (Isovue™) are suitable contrast agent which can be used safely and efficiently with the methods and kits of the current disclosure.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

1. A method of delivering a composition comprising a bioagent to a targeted area of an organ comprising:
a) determining an effective total volume for injection of the composition, wherein said determination of the total volume for injection is based on the compliance of tissue in the targeted area;
b) preparing the composition comprising the bioagent, a contrast agent, and a vehicle, in about the total volume determined in step (a), wherein said composition has a final osmolarity from about 250 mOsm to about 440 mOsm;
c) administering at least a portion of the composition prepared in step (b) in volumes that optimizes a retention of the bioagent in the targeted area while minimizing systemic distribution; and wherein said method provides an administrator with intra-operative feedback.

2. The method of claim 1, wherein the step of dispensing at least a portion of said suspension into the targeted area comprises at least one injection of at least a portion of the suspension into the targeted area.

3. The method of claim 1 wherein the contrast agent is selected from the group consisting of iodine-based compounds, gadolinium-based compounds, and any combination thereof.

4. The method of claim 1 wherein the contrast agent comprises iopamidol.

5. The method of claim 1 wherein the contrast agent comprises iodixanol.

6. The method of claim 1, wherein the contrast agent is present in the suspension at a concentration from about 25% v/v to about 35% v/v.

7. The method of claim 1, wherein the vehicle is selected from a group consisting of water, culture media, or cell-friendly shipping media.

8. The method of claim 1, wherein the bioagent is selected from the group consisting of cells, proteins, drugs, nucleic acids, or a combination thereof.

9. The method of claim 8, wherein the cells are selected from the group consisting of skeletal myocytes, cardiomyocytes, purkinje cells, fibroblasts, myoblasts, mature endothelial cells, mature epithelial cells, hematopoietic cells, adult stem cells, embryonic stem cells, pluripotent stem cells, mesenchymal stem cells, endodermal stem cells, ectodermal stem cells, islet cells, hepatocytes, chondrocytes, osteoblasts, neuronal cells, glial cells, smooth muscle cells, endothelial cells, skeletal myoblasts, nucleus pulposus cells, epithelial cells, and any combination thereof.

10. The method of claim 9, wherein the adult stem cells are derived from a source selected from the group consisting of brain, bone marrow, peripheral blood, cord blood, blood vessels, skeletal muscle, skin liver, and heart.

11. The method of claim 9, wherein at least a portion of the cells is modified with an extraneous genetic material.

12. The method of claim 11, wherein the extraneous genetic material comprises at least one nucleotide sequence capable of an alteration of a phenotype of a member of at least the portion of the cells.

13. The method of claim 12, wherein the alteration of the phenotype of the member of at least the portion of the cells includes an alteration of expression or activity of at least one gene.

14. The method of claim 13, wherein the at least one gene encodes a protein selected from the group consisting of proteins mediating cell survival, proteins mediating cell attachment, cardiomyocyte markers, and any combination thereof.

15. The method of claim 1, wherein the bioagent is selected from a group consisting of biologic or synthetic compounds selected from the group consisting of anti-inflammatory compounds, anti-proliferative compounds, anti-bacterial compounds, pro-cell survival compounds, analgesic compounds, and any combination thereof.

16. The method of claim 1, wherein the suspension is delivered in a plurality of injections.

17. The method of claim 16, wherein a volume of a member of a plurality of injections is between about 10 μl and about 200 μl.

18. The method of claim 17, wherein the volume of the member of the plurality of injection is customized depending on the compliance of the target tissue between about 10 μl and about 160 μl.

19. The method of claim 18, wherein the volume of the member of the plurality of injection is between about 10 μl and about 80 μl.

20. A method of delivering a bioagent to a targeted area of an organ comprising:
a) preparing a suspension comprising the bioagent, a contrast agent, a vehicle, wherein said suspension has a final osmolarity from about 250 mOsm to about 440 mOsm, wherein the total volume of the suspension injected into a targeted area is directly proportional to an interstitial capacity, and is determined by an equation: tVol=LVim×ISc, wherein tVol is a total volume for injection, LVim is a LV Infarcted mass(g), and ISc is Interstitial capacity (ml/g);
b) providing the operator with intra-operative feedback; and
c) dispensing at least a portion of said suspension into the targeted area.

21. The method of claim 20, wherein the interstitial capacity of the targeted area is between about 0.08 ml/g and about 0.43 ml/g.

22. The method of claim 21, wherein the interstitial capacity of the targeted area is between about 0.12 ml/g and about 0.20 ml/g.

23. The method of claim 21, wherein the organ allows a minimally invasive access.

24. The method of claim 21, wherein the organ is a heart.

25. The method of claim 24, wherein the targeted area is a myocardial region.

26. The method of claim 24, wherein the targeted area is selected from the group consisting of intraventricular septum, apex, left ventricle free wall, left ventricle lateral wall, left ventricle posterior wall, and any combination thereof.

27. The method of claim 24, wherein the bioagent is selected from the group consisting of cells, proteins, drugs, nucleic acids, or a combination thereof.

28. The method of claim 27, wherein the cells are selected from the group consisting of skeletal myocytes, cardiomyocytes, Purkinje cells, fibroblasts, myoblasts, mature endothelial cells, mature epithelial cells, hematopoietic cells, adult stem cells, embryonic stem cells, pluripotent stem cells, mesenchymal stem cells, endodermal stem cells, ectodermal stem cells, islet cells, hepatocytes, chondrocytes, osteoblasts, neuronal cells, glial cells, smooth muscle cells, endothelial cells, skeletal myoblasts, nucleus pulposus cells, epithelial cells, and any combination thereof.

29. The method of claim 28, wherein the adult stem cells are derived from a source selected from the group consisting of brain, bone marrow, peripheral blood, cord blood, blood vessels, skeletal muscle, skin, liver, and heart.

30. The method of claim 28, wherein at least a portion of the cells is modified with an extraneous genetic material.

31. The method of claim 30, wherein the extraneous genetic material comprises at least one nucleotide sequence capable of an alteration of a phenotype of a member of at least the portion of the cells.

32. The method of claim 31, wherein the alteration of the phenotype of the member of at least the portion of the cells includes an alteration of expression or activity of at least one gene.

33. The method of claim 32, wherein the at least one gene encodes a protein selected from the group consisting of proteins mediating cell survival, proteins mediating cell attachment, cardiomyocyte markers, and any combination thereof.

34. The method of claim 24, wherein the bioagent is selected from a group consisting of biologic or synthetic compounds selected from the group consisting of anti-inflammatory compounds, anti-proliferative compounds, anti-bacterial compounds, pro-cell survival compounds, analgesic compounds, and any combination thereof.

35. The method of claim 24, wherein at least a portion of the cells comprises a marker.

36. The method of claim 35, wherein the marker comprises superparamagnetic iron.

37. The method of claim 24, wherein the targeted area is a localized ischemic lesion.

38. The method of claim 37, wherein the total volume of the suspension injected into the blood vessel approximately equals a product of the volume of the targeted area and the interstitial capacity, wherein the interstitial capacity is between about 0.08 ml/g and about 0.43 ml/g.

39. The method of claim 38, wherein the targeted area is a chronic, noncalcified ischemic lesion and the interstitial capacity of the targeted area is between about 0.12 ml/g and about 0.20 ml/g.

40. The method of claim 38, wherein the step of dispensing at least a portion of said suspension into the targeted area comprises at least one injection of at least a portion of the suspension into the targeted area.

41. The method of claim 40, wherein the step of dispensing at least a portion of said suspension into the targeted area further comprises at least a second injection of at least a portion of the suspension into the targeted area and wherein the a distance between the at least one injection and at least the second injection is at least about 3 mm.

42. The method of claim 1, wherein the suspension is delivered via a catheter.

43. The method of claim 42, wherein the catheter is a minimally invasive, percutaneous, transvenous catheter.

44. The method of claim 1, wherein the contrast agent provides a real-time topographic and delivery guidance.

45. A kit for delivering a bioagent into a targeted area of an organ comprising:
a delivery device;
a bioagent;
a contrast agent;
a vehicle; and
a set of instructions.

46. The kit of claim 45, wherein the combination of the bioagent, the contrast agent and the vehicle has an osmolarity between about 250 mOsm and 350 mOsm.

47. The kit of claim 45, wherein the contrast agent is selected from the group consisting of iodine-based compounds, gadolinium-based compounds, and any combination thereof.

48. The kit of claim 45, wherein the contrast agent comprises iopamidol.

49. The kit of claim 45, wherein the contrast agent comprises iodixanol.

50. The kit of claim 45, wherein the contrast agent is present in the suspension at a concentration from about 25% v/v to about 35% v/v.

51. The kit of claim 45, wherein the vehicle is selected from a group consisting of water, culture media, or cell-friendly shipping media.

52. The kit of claim 45, wherein the bioagent is selected from the group consisting of cells, proteins, drugs, nucleic acids, or a combination thereof.

53. The kit of claim 52, wherein the cells are selected from the group consisting of skeletal myocytes, cardiomyocytes, Purkinje cells, fibroblasts, myoblasts, mature endothelial cells, mature epithelial cells, hematopoietic cells, adult stem cells, embryonic stem cells, pluripotent stem cells, mesenchymal stem cells, endodermal stem cells, ectodermal stem cells, islet cells, hepatocytes, chondrocytes, osteoblasts, neuronal cells, glial cells, smooth muscle cells, endothelial cells, skeletal myoblasts, nucleus pulposus cells, epithelial cells, and any combination thereof.

54. The kit of claim 53, wherein the adult stem cells are derived from a source selected from the group consisting of brain, bone marrow, peripheral blood, cord blood, blood vessels, skeletal muscle, skin, liver, and heart.

55. The kit of claim 52, wherein at least a portion of the cells is modified with an extraneous genetic material.

56. The kit of claim 55, wherein the extraneous genetic material comprises at least one nucleotide sequence capable of an alteration of a phenotype of a member of at least the portion of the cells.

57. The kit of claim 56, wherein the alteration of the phenotype of the member of at least the portion of the cells includes an alteration of expression or activity of at least one gene.

58. The kit of claim 57, wherein the at least one gene encodes a protein selected from the group consisting of proteins mediating cell survival, proteins mediating cell attachment, cardiomyocyte markers, and any combination thereof.

59. The kit of claim 52, wherein the bioagent is selected from a group consisting of biologic or synthetic compounds selected from the group consisting of anti-inflammatory compounds, anti-proliferative compounds, anti-bacterial compounds, pro-cell survival compounds, analgesic compounds, and any combination thereof.

60. The kit of claim 45, further comprising a marker.

61. The kit of claim 60, wherein the marker is selected from the group consisting of europium nanoparticles, superparamagnetic iron oxide, and any combination thereof.

62. The kit of claim 60, wherein the bioagent comprises a plurality of cells and wherein at least a portion of said plurality of cells is labeled with a marker.

63. The kit of claim 45, wherein the delivery device is a catheter.

64. The kit of claim 63, wherein the catheter is a percutaneous transvenous catheter.

65. The kit of claim 45, wherein the set of instruction comprises information on preparation of a suspension comprising the bioagent, the contrast agent, and the vehicle, wherein the total volume to be injected into the targeted area is based on the compliance of tissue in the targeted area.
